(19) United States
(12) Patent Application Publication
(10) Pub. No.: US 2018/0311404 A1
(43) Pub. Date: Nov. 1, 2018

(54) DEVICE FOR PREPARING A BIOLOGICAL WOUND DRESSING MADE OF AUTOLOGOUS FIBRIN

(71) Applicant: Hélio Nogueira da SILVA JUNIOR, Juiz de Fora (BR)

(72) Inventor: Hélio Nogueira da SILVA JUNIOR, Juiz de Fora (BR)

(21) Appl. No.: 15/771,260

(22) PCT Filed: Oct. 27, 2015
(86) PCT No.: PCT/BR2015/050192

§ 371 Date: Apr. 27, 2018

(51) Int. Cl. A61L 24/10; A61M 1/36

(52) U.S. Cl. CPC A61L 24/106; A61M 1/3693

(57) ABSTRACT

A device for preparing a biological wound dressing made of autologous fibrin. The device includes a cylindrical container with a bottom which is inclined from the side slopes towards a central channel with a slope that leads the liquid sealant remaining from pressing the fibrin clot through the sieve towards the outlet and from there to the multiduct collection system.

### BRIEF PRESENTATION

This application for Patent of Invention concerns an original disposable device manufactured from material biocompatible with human blood, constituting a closed system which, due to its specificities, allows the squeezing of a fibrin clot, resulting in the separation of the solid sealant from the liquid sealant, thus creating an autologous curative, that is, one which uses the blood of the patient itself.

### FIELD OF APPLICATION

The device is used by professionals from the health field in a broad range of procedures, elective or otherwise, which may take place in health centers, doctors' or dentists' clinics, highly complex hospital facilities, first-aid centers, basic health units, and even in remote places with limited infrastructure, such as combat zones, oil rigs, rural areas etc.

### CONTEXTUALIZATION

Blood is a fluid tissue, formed of a cellular portion which circulates in suspension in a liquid medium, known as plasma. This is composed of 92% water and the remaining 8% of proteins, salts and other organic components in dissolved form. The cellular phase is composed of red blood cells (erythrocytes), white blood cells (leucocytes) and platelets which are incomplete cells formed only of portions of the cytoplasm of the cells from which they originate (mega karyocytes).

The platelets participate in the coagulation process as well as play an active role in repairing wounds, being the first components present at the site of trauma, and possess anti-inflammatory and regenerative properties. Once activated, the platelets release growth factors.

In this context the biological sealant is the liquid part plus the Platelet Rich Plasma (PRP), and reproduces the final phase of blood coagulation, where the fibrinogen is converted into fibrin in the presence of thrombin (factor XIII), fibronect in and ionized calcium (plasma proteins). The thrombin and fibrinogen promote the sealing of the surgical area.

### STATE OF THE ART

Generally speaking, the creation of a biological curative is impossible in autologous form, but only in heterologous form using blood collected from third parties.

This is because the most commonly used resource for performing this procedure is wholly improvised, involving a high degree of handling by the professional, and because it possesses components made of stainless steel, with an acid pH, it is not biocompatible with human blood, which is mildly alkaline.

Operationally speaking, the product deriving from a blood bank is subjected to a centrifugation process in order to obtain a fibrin clot. By using tweezers and scissors, the PRP is cut over a stainless [steel] sieve and set aside in a separate container, made of the same material. Then, the PRP is squeezed over a stainless [steel] plate possessing orifices which drain the liquid part into a container with a flat bottom.

In brief, the preparation of a curative with a sealant as described herein presents the following main drawbacks:

It renders unviable the preparation of the curative in autologous form;

It is impossible to standardize the form and consistency of the curative—a manual process, involving the application of different compressive forces;

Risk of contamination due to the fact that it is an open process;

Waste—the liquid left over from the compression is lost, as the container in which it is deposited has a flat bottom, lacking the possibility of channeling, draining or storing the fluid;

The efficacy of the product is compromised—the difference in the hydrogenionic potential of the stainless steel and the blood causes the partial neutralization of the clot, inactivating a substantial portion of the final product.

The current state of the art anticipates some patent documents concerning autologous means of preparing curatives, such as document US 20050236325 which comprises a centrifuge with a reservoir to receive and separate a patient's blood sample, forming and separating platelet rich plasma from the platelet poor plasma and red blood cells. An activating agent is added in the first chamber and forms a clot which is ground, and the resulting serum containing autologous thrombin is collected. This is mixed in the second chamber with the platelet rich plasma forming the sealant. The above equipment, despite allowing for the autologous preparation of the curative, involves a complex construction process and complicated asepsis/cleaning aspects.

This limits its broad use and use in locations lacking infrastructure.

### OBJECTIVES OF THE INVENTION

A primary objective is to provide a disposable device, manufactured from material that is biocompatible with human blood, that is easy to use and which possess great mobility, allowing the preparation of consistent and homogeneous curatives based on fibrin extracted from the patient's own blood.

A second objective is to provide for the complete collection and use of the liquid part remaining from the squeezing of the fibrin clot.

A third objective is to produce a curative of high quality and effectiveness.

### SUMMARY OF THE INVENTION

The device is composed of a cylindrical bottom container with slopes directed towards a central channel with a posteroanterior drop, which ends in a nozzle connected to the hoses and syringes of a multi-path collection system. A sieve possessing orifices for insertion within the container serves as a base for squeezing the fibrin clot created in a centrifuge using the patient's own blood. The lid of the device exerts light pressure on the fibrin clot removing the excess liquid that passes through the orifices and, as previously mentioned, is drained through the central channel in the direction of a multi-path collection system. Thus, by squeezing the clot, it is possible to prepare a consistent and homogeneous curative ready for use, with total preservation of the liquid sealant. In the sieve some orifices of greater diameter stand out which serve to receive conical molds, of the thimble type, which, when filled with the fibrin clot squeezed with a dowel pin, constitute a curative with a differentiated form appropriate for use, for example, in the dental implant segment.

Below, the invention is explained with reference to the attached designs for non-limiting, illustrative purposes, where the following are represented:

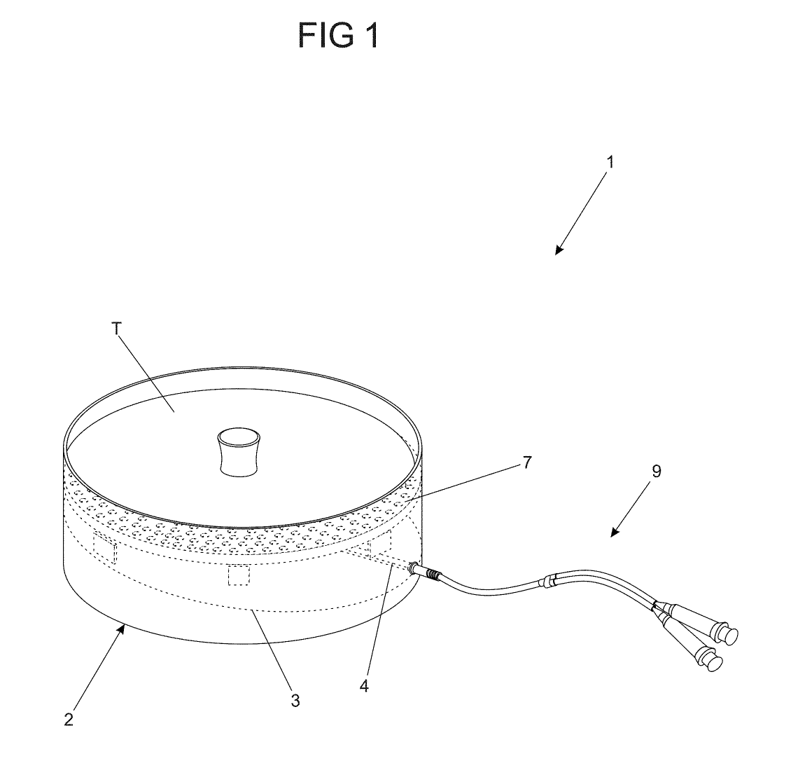

FIG. 1: Perspective view of the Device for Preparing a Biological Wound Dressing Made of Autologous Fibrin;

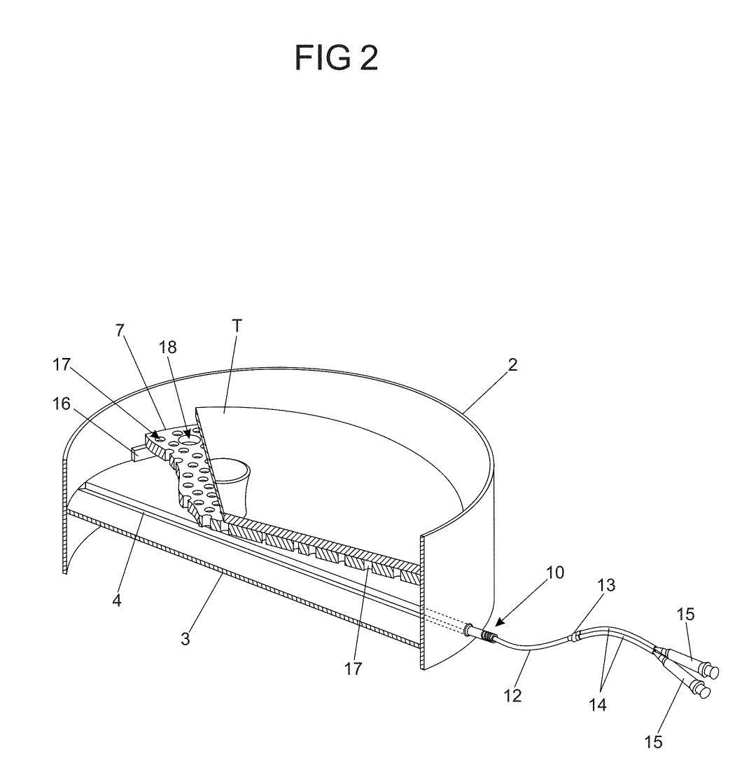

FIG. 2: Partial cross-section perspective view of the Device for Preparing a Biological Wound Dressing Made of Autologous Fibrin;

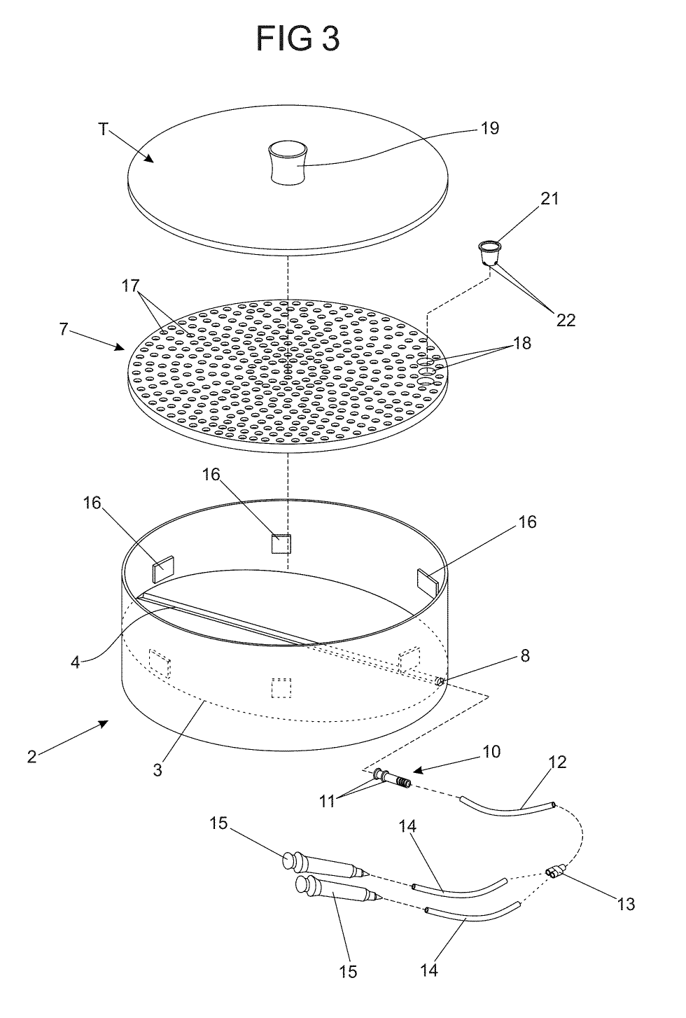

FIG. 3: Exploded perspective view of the Device for Preparing a Biological Wound Dressing Made of Autologous Fibrin;

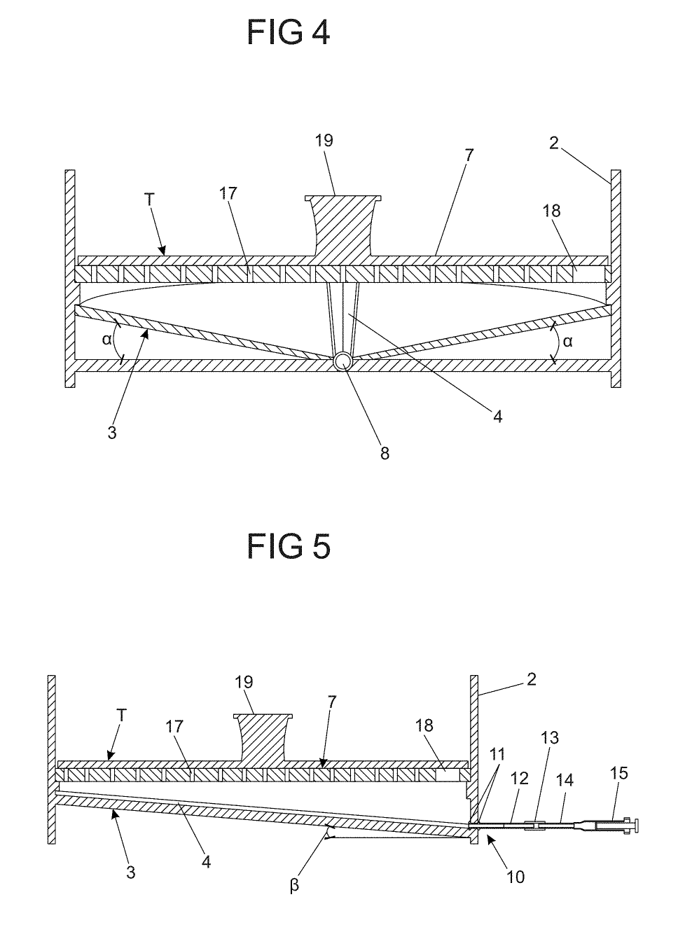

FIG. 4: Front cross-sectional view of the Device for Preparing a Biological Wound Dressing Made of Autologous Fibrin;

FIG. 5: Side cross-sectional view of the Device for Preparing a Biological Wound Dressing Made of Autologous Fibrin;

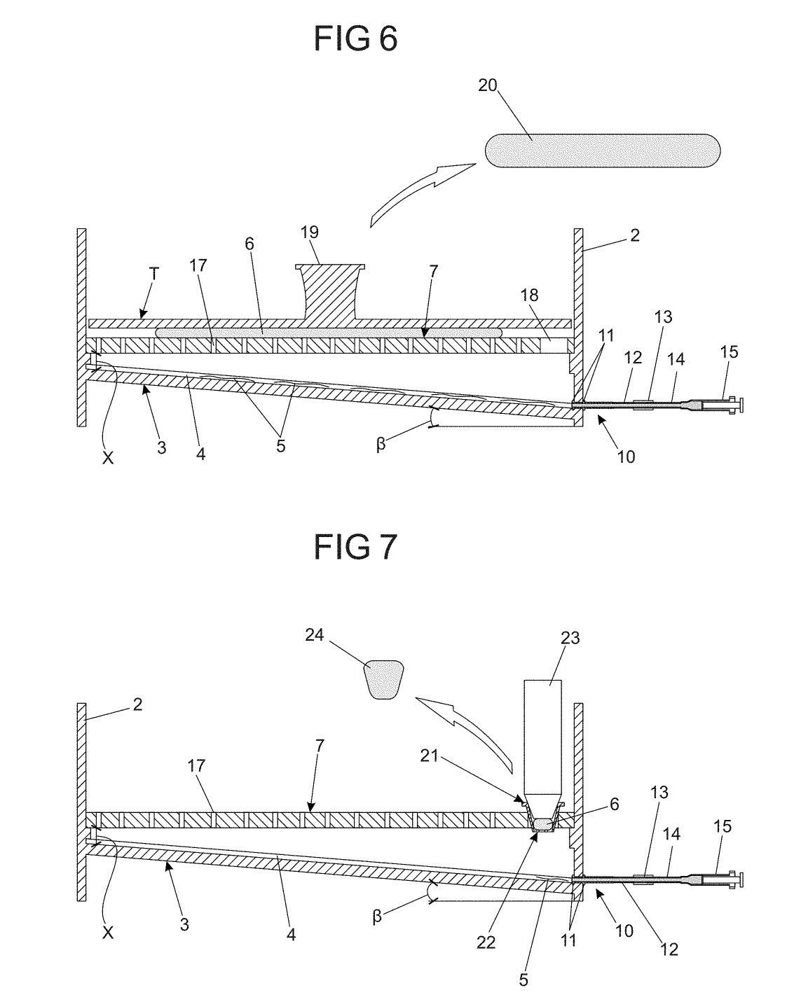

FIG. 6: Side cross-sectional view of the Device for Preparing a Biological Wound Dressing Made of Autologous Fibrin, showing use and curative created;

FIG. 7: Side cross-sectional view of the Device for Preparing a Biological Wound Dressing Made of Autologous Fibrin, showing use with mold and alveolar curative created.

### DETAILED DESCRIPTION OF THE INVENTION

The Device for Preparing a Biological Wound Dressing Made of Autologous Fibrin consists of a device (1) composed of a cylindrical container (2) with a bottom (3) with side slopes (α) directed towards a central channel (4) with a slope (β), which directs the liquid sealant (5), left over from the pressing of the fibrin clot (6) through a sieve (7), in the direction of the exit nozzle (8), and thence to the multi-path collecting system (9).

More specifically, the device (1) is manufactured from material that is biocompatible with human blood, preferably polypropylene sterilized with ethylene oxide, and is composed of a cylindrical container (2) with a bottom (3) with side slopes (α) directed towards a central channel (4) with a slope (β), ending in a nozzle (8) which receives a tip (10) with latches (11), ensuring fastening to said container (2). A multi-path collecting system (9) originates from the tip and it consists of at least one hose (12) connected to a valve (13) with at least two pathways, each connected by hoses (14) to syringes (15), where the liquid sealant (5) left over from the squeezing of the fibrin clot (6) is stored, thus allowing its full use. On the inner wall of the container(2) there are perimeter stoppers (16) that serve as bases to support the sieve (7) with two diameters of orifices (17 and 18), maintaining a space (X) in relation to the bottom (3), sufficient for the draining of the liquid sealant (5) in the direction of the multi-path collection system (9). For this purpose, the fibrin clot (6) is placed over the sieve (7) and respective orifices (17), and subjected to light compression exerted by the lid (T) whose handle (19), design and dimensions allow the internal drainage into the container (2), creating a standardized, homogeneous curative (20) with symmetry of size and thickness. Finally, the larger orifices (18) are designed to receive conical molds (21) whose ends possess orifices (22) for the drainage of the liquid sealant (5) left over from the pressing of the fibrin clot (6) produced by an appropriate dowel pin (23), thus creating an alveolar curative (24). Similarly, the remaining liquid sealant (5) is captured by the multi-path collecting system (9), and fully used.

For the reasons provided above, the original disposable device made from material biocompatible with human blood exhibits a high degree of novelty since it enables the autologous preparation of the curative, without any kind of loss/waste of the biological sealants that flow through a sterile circuit in a closed system without margin for contamination which, added to its utility, makes it deserving of the patent privilege.

1-5. (canceled)

6. A device for preparing a biological wound dressing made of autologous fibrin, made from material biocompatible with human blood, comprising:
a container with a bottom with side slopes and a channel with a drop in a direction of a nozzle; and
a stopper support a sieve with orifices of two diameters beneath a lid for pressing a fibrin clot made of a patient's own blood, which flows through the orifices to a multi-path collecting system.

7. The device for preparing a biological wound dressing made of autologous fibrin, according to claim 6, further comprising a multi-path system deriving from a tip with latches, formed of at least one hose connected to a valve with at least two pathways connected by hoses to syringes for suction of the liquid sealant.

8. The device for preparing a biological wound dressing made of autologous fibrin, according to claim 6, further comprising an orifice for receiving conical molds with orifices.

9. The device for preparing a biological wound dressing made of autologous fibrin, according to claim 8, further comprising a squeezing of the fibrin clot in the molds by a dowel pin.

10. The device for preparing a biological wound dressing made of autologous fibrin, according to claim 8, further comprising the creation of a curative through the use of orifices, and alveolar curatives through the use of molds in the orifices.
